(19) United States
(12) Patent Application Publication
(10) Pub. No.: US 2007/0287904 A1
(43) Pub. Date: Dec. 13, 2007

(54) Method and apparatus for determining a hemodynamic response function for event-related functional magnetic resonance imaging

(76) Inventors: Yu Li, Gainesville, FL (US); Mark Limkeman, Gainesville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/788,586

(22) Filed: Apr. 20, 2007

Related U.S. Application Data
(60) Provisional application No. 60/794,049, filed on Apr. 20, 2006.

(51) Int. Cl. A61B 5/055

(52) U.S. Cl. 600/410

(57) ABSTRACT

Embodiments of the subject invention can involve a method of suppressing noise in hemodynamic deconvolution for event-related functional MR imaging (ER-fMRI). A typical ER-fMRI experiment measures the Blood Oxygenation Level Dependent (BOLD) response to a series of sparse, short-duration stimuli. Based on the deconvolution of a hemodynamic response function (HRF) from the BOLD signal and event stimulus function, the neuronal activation can be localized to specific brain regions and tracked on the order of a second. ER-fMRI can be used to study the temporal dynamics of neuronal network. However, in certain situations, aliasing noise can be generated in hemodynamic deconvolution due to the low sampling rate limited by the imaging speed. This aliasing noise can reduce the accuracy of temporal characterization of the HRF. In an embodiment, by incorporating the use of a phantom having one or more coil loops positioned perpendicular to the magnetic field Bₒ, such that DC current inputted into one of the loops will produce field distortion to Bₒ, an ER-fMRI experiment can be calibrated and the temporal measurement of HRF can be improved with the removal of aliasing noise.

### CROSS-REFERENCE TO RELATED APPLICATION

The present application claims the benefit of U.S. Provisional Patent Application Ser. No. 60/794,049, filed Apr. 20, 2006, which is hereby incorporated by reference herein in its entirety, including any figures, tables, or drawings.

### BACKGROUND OF INVENTION

In event-related functional MRI, there exist limits on the time length of the experiments on human subjects and the imaging speed. Due to these limitations, data truncation and undersampling have to be used in functional MRI signal acquisition. It is observed that the high frequency components generated due to data truncation can fold back into low frequencies when the sampling rate is not sufficiently high. This aliasing can introduce significant noise in hemodynamic deconvolution and can reduce the accuracy of the temporal characterization of hemodynamic response.

An event-related functional magnetic resonance imaging (ER-fMRI) experiment measures the blood oxygen level dependent (BOLD) response to a series of sparse, short-duration stimuli. This allows the deconvolution of an impulse response function assuming that the BOLD fMRI is a linear time-invariant (LTI) system (Buckner et al., 1996, 1998; Rosen et al., 1998; Boynton et al., 1996; Glover, 1999; Liu et al., 2001; Zarahn, 2000). It is understood that the BOLD response is coupled to a set of physiological responses, which mainly include cerebral blood flow (CBF), cerebral blood volume (CBV), and cerebral metabolic rate of oxygen (CMRO₂). In the literature, these physiological responses are referred to collectively as hemodynamic response and the resolved impulse response in ER-fMRI is termed as hemodynamic response function (HRF) (Buckner et al., 1996; Ritchter et al., 1997; Friston et al., 1998; Menon et al., 1998; Dilharreguy et al., 2003). It is believed that the dynamic change of CBF, CBV and CMRO₂ in neuronal activation determines the basic shape of HRF, which often presents an initial dip, a peak, and a post-stimulus undershoot sequentially over the entire time evolution of about 20 s (Buxton et al., 1998, 2004; Ernst et al., 1994; Hu et al., 1997; Menon et al., 1995). A primary goal of ER-fMRI is to specify the temporal behavior of underlying neuronal activity based on the measurement of the temporal parameters of HRF, such as time-to-onset, time-to-peak, and time-to-undershoot. Furthermore, by comparing these temporal parameters across regions in the brain, it is possible to track the neuronal activity in the spatio-temporal domain. This can provide means to study the temporal dynamics of the neuronal network, to analyze the cognitive process, and to understand the brain function (Buckner et al., 1996; Dilharreguy et al., 2003; Friston et al., 1998; Kruggel et al., 1999; Neumann et al., 2003; Ritchter et al., 1997). However, there are many technical challenges remaining to be solved regarding the temporal characterization of HRF. One such challenge reported recently is the substantial variation of HRF across individuals for a given brain region or across regions for the same individual (Buxton et al., 2004; Miezin et al., 2000; Dilharreguy et al., 2003; Neumann et al., 2003; Handwerker et al., 2004; Duann et al., 2002; Liu et al., 2001). Due to this variation, the ability of ER-fMRI to track the neuronal activity strongly depends on how accurately the temporal parameters of HRF can be measured in the different regions of the brain.

Practically, in an ER-fMRI experiment, the accuracy of the temporal characterization of HRF is limited by the imaging speed. An optimized echo-planar imaging (EPI) technique at 3 T without the use of parallel acquisition, needs the minimum transient repetition time (TR) to be typically 1˜2 s for the full coverage of brain. This low sampling rate (long TR) imposes a limit on the temporal resolution of HRF deconvolution. Several recent papers have suggested that this limitation could be overcome in certain cases using averaging or interpolation methods or HRF model fitting (Kruggel et al., 1999; Miezin et al., 2002, Friston et al., 1998; Menon et al., 1998). However, some inconsistency regarding the temporal precision of HRF measurements was found in different experiments and the efficiency of these methods is unknown (Miezin et al. 2000; Maccotta et al. 2001). The influence of fMRI data sampling on temporal characterization of HRF was extensively investigated by Dilharreguy et al., 2003. It was demonstrated that the accuracy of HRF measurements can be affected by many parameters in data sampling and model fitting. This suggests that the improvement of the temporal accuracy in HRF measurements is very difficult when a high sampling rate is not achievable. To date, a major conclusion that has been made in most of the studies is that the temporal accuracy of HRF measurements increases with sampling rate. Many issues regarding the influence of data sampling on the temporal characterization of HRF in ER-fMRI still remain unclear and a general solution to improve the temporal accuracy of HRF measurements at a low sampling rate is not yet available.

A basic assumption for HRF deconvolution is that the sampled data can be modeled as a LTI system. Although this model has been used in many experimental data analysis, the validity of the above assumption has never been conclusively established. (Boynton et al., 1996; Glover, 1999; Buxton et al., 2004; Kershaw et al. 2001). Moreover, the observation of nonlinearity in ER-fMRI data has been reported in several studies (Buxton et al., 2004; Friston and Josephs et al., 1998; Kershaw et al., 2001; Vazquez et al., 1998). In one of the earliest work on system modeling of fMRI data, Boynton et al. (1996) considered a model with three cascaded units: the neuronal pathway, the hemodynamics, and the imaging system. In most of the later ER-fMRI studies, this model has been simplified by neglecting the neuronal pathway and the imaging system. The HRF deconvolution is generally based on a LTI model shown below (Glover, 1999; Friston et al., 1998; Burock et al., 2000; Lu et al., 2005; Dale, 1999; Gitelman et al., 2003)

s(t)=f(t)\textcircled{×}h(t)+n(t), (1)

where s(t) is the time series of image intensity (BOLD signal), f(t) represents the event stimulus function, h(t) is the HRF, and n(t) is the noise. Neglecting the imaging system may result in the reduction of accuracy of the temporal characterization of HRF in ER-fMRI analysis, when the sampling frequency is low. As the BOLD response is slow, the signal components should preferably be within a low and narrow frequency band in frequency domain. In such a frequency band, the imaging system is expected to perform like a linear and all-pass system. If only the temporal behavior is of concern, Eq. (1) appears to be a good approximation of ER-fMRI data model. This is true only if the sampling rate is sufficiently high. However, fMRI data is usually acquired at a low sampling rate due to the limited imaging speed, as mentioned earlier. This undersampling can introduce some nonlinear aliasing effects in the BOLD signal acquisition, because high frequency components, if present, can fold back into the low frequencies. Unfortunately, in BOLD fMRI experiments, sources that are able to generate such high frequency components are present. The data is typically acquired within a limited time length because human subjects do not wish to have a lengthy stay inside the magnet and a short experiment time is commonly preferred. The data truncation in time domain is equivalent to the convolution with a SINC function in frequency domain. This will result in some signal leakage from low frequencies to high frequencies. The shorter the data length, the more signal leak there is. Therefore, nonlinear distortion may be introduced in HRF deconvolution for ER-fMRI due to the aliasing. If this distortion affects the temporal characterization of HRF considerably, Eq. (1) may not serve as an adequate model for ER-fMRI. Accordingly, there is a need for a method and apparatus for determining a hemodynamic response function for event-related functional magnetic resonance imaging that can address aliasing effects.

### DETAILED DESCRIPTION OF THE FIGURES

FIG. 1 shows an experimental set up for fMRI calibration using a BOLD simulator in accordance with a specific embodiment of the subject invention.

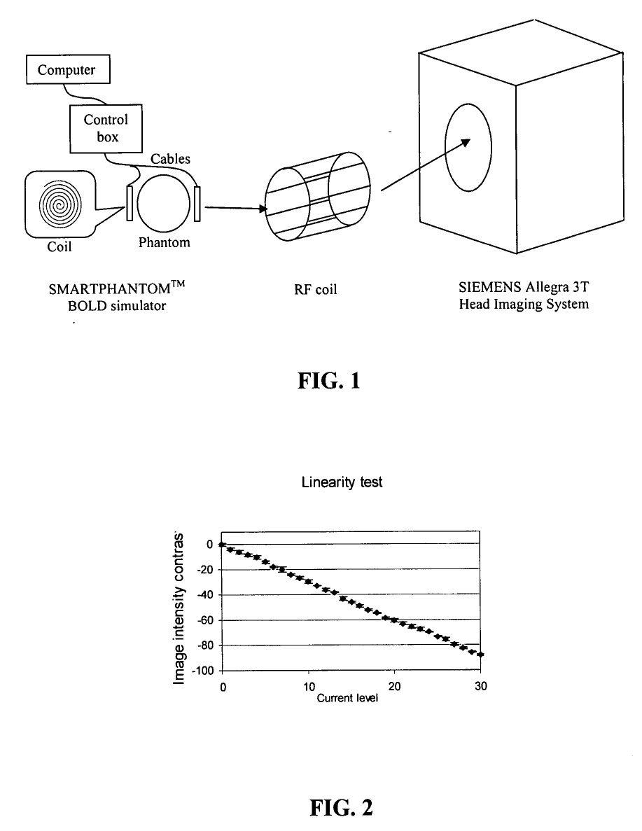

FIG. 2 shows a linearity test plot of the BOLD simulator of FIG. 1, where image intensity contrast is plotted along the vertical axis against current level along the horizontal axis, and mean values and standard errors of the measured image intensity contrast are given.

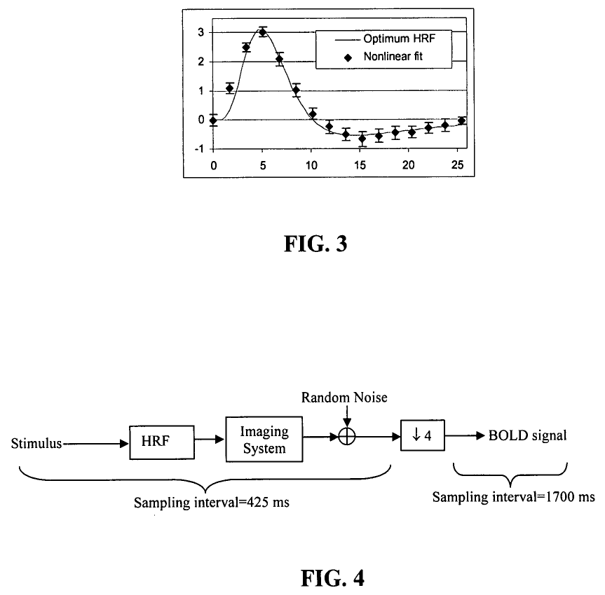

FIG. 3 shows a determination of the standard HRF using a nonlinear fitting method, where the solid line is the nonlinear fitting result given by Equation (3), and the data points of the optimum HRF obtained by averaging 50 resolved HRFs are denoted by “♦”, with mean values and standard errors given.

FIG. 4 shows a system model used in simulation in accordance with the subject invention.

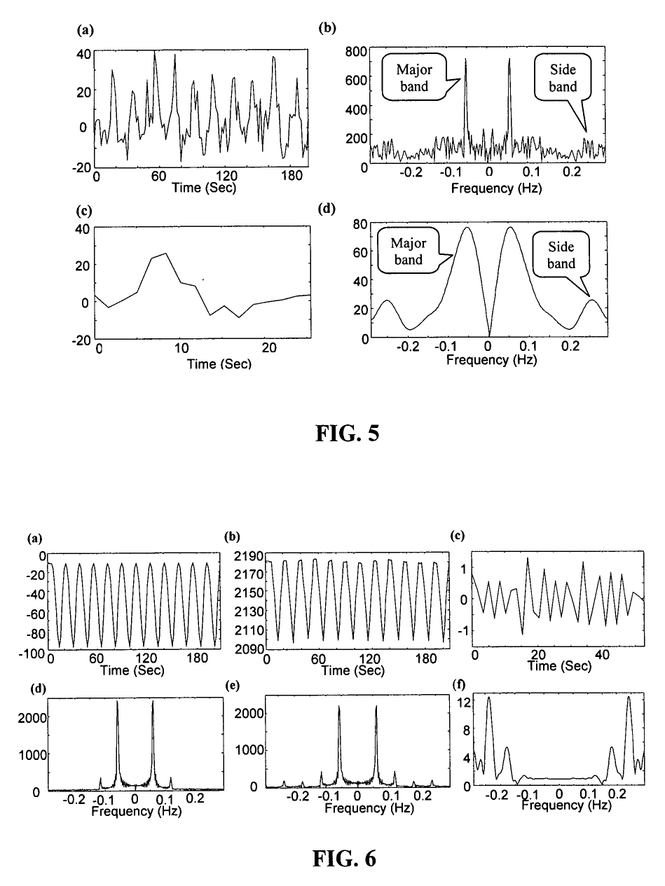

FIGS. 5A-5D show a BOLD signal acquired from a voxel of subject #1 and the HRF resolved from this signal, where FIG. 5A shows a BOLD signal; FIG. 5B shows a Fourier transform of BOLD signal; FIG. 5C shows a resolved HRF; and FIG. 5D shows a Fourier transform of HRF.

FIGS. 6A-6F shows a determination of an aliasing transfer function for an ER-fMRI experiment on a human subject #1, where: FIG. 6A shows the time course of current signal input to the BOLD simulator with aliasing removed; FIG. 6B shows the time course of imaging intensity in the calibration scans; FIG. 6C shows an aliasing transfer function in time domain; and FIGS. 6D-6F show the Fourier transforms of FIGS. 6A-6C.

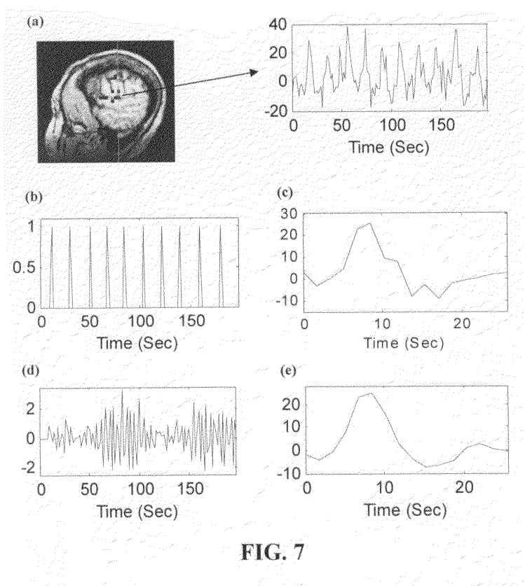

FIGS. 7A-7E show HRF deconvolution using direct deconvolution and an anti-aliasing embodiment in accordance with an embodiment of the subject method, where: FIG. 7A shows a BOLD signal from subject #1; FIG. 7B shows an event stimulus function; FIG. 7C shows an HRF resolved by direct deconvolution; FIG. 7D shows the convolution of event stimulus function and aliasing transfer function; and FIG. 7E shows an HRF resolved by anti-aliasing method.

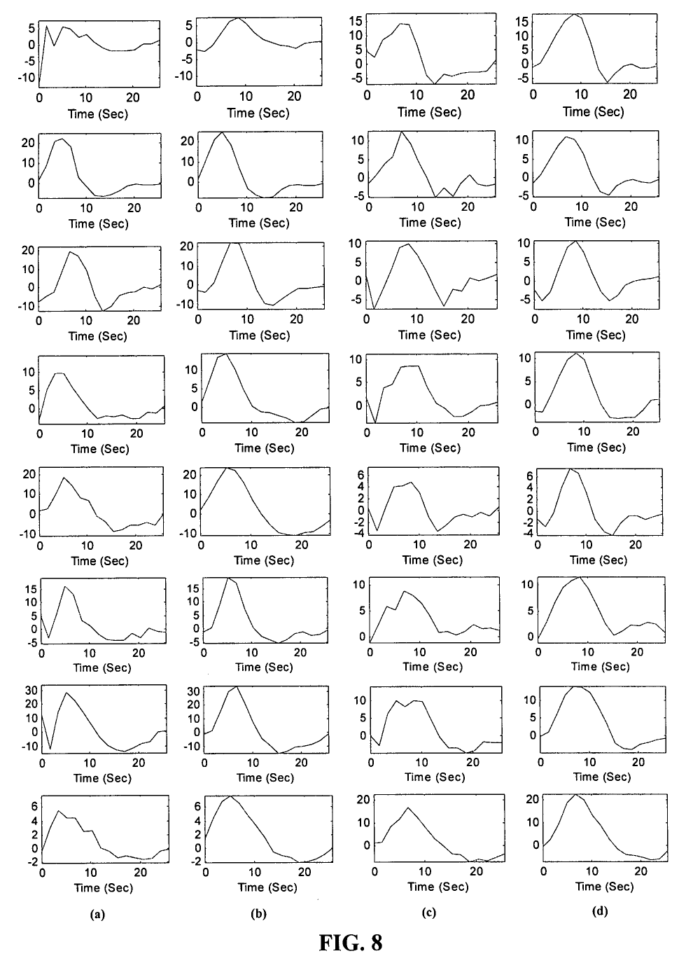

FIGS. 8A-8D show HRF an deconvolution, where FIG. 8A shows an HRF deconvolution for voxels in the motor cortex of eight human subjects using direct deconvolution; FIG. 8B shows HRF deconvolution for voxels in the motor cortex of eight human subjects using an anti-aliasing embodiment in accordance with the subject method; FIG. 8C shows an HRF deconvolution for voxels in vision cortex of eight human subjects using direct deconvolution; and FIG. 8D shows an HRF deconvolution for voxels in vision cortex of eight human subjects using an anti-aliasing embodiment in accordance with the subject method.

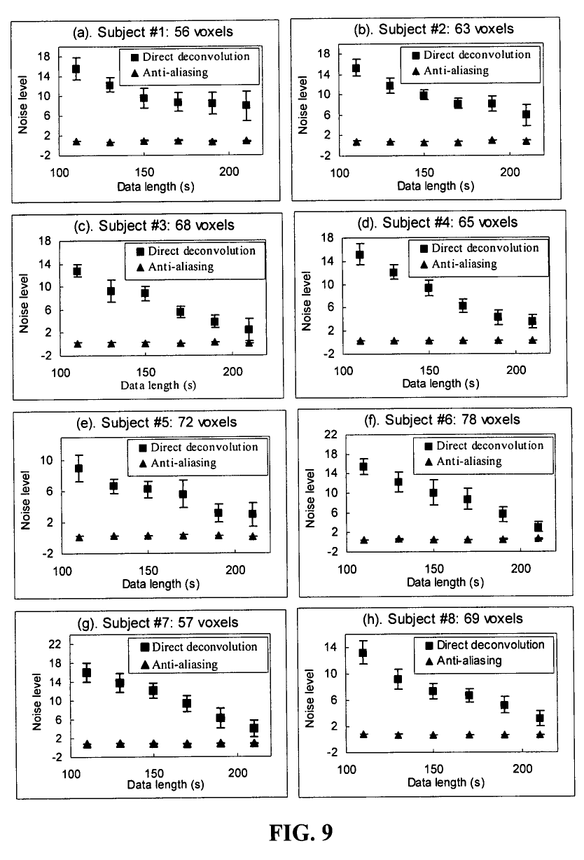

FIG. 9 shows quantitative analysis of noise suppression, with the noise level plotted along the vertical axis against the data length along the horizontal axis, where the noise level is defined as the power of the frequency components between 0.2 and 0.4 Hz in percent of the total power of the HRF and mean values and standard errors of the statistic measurements on every subject are given.

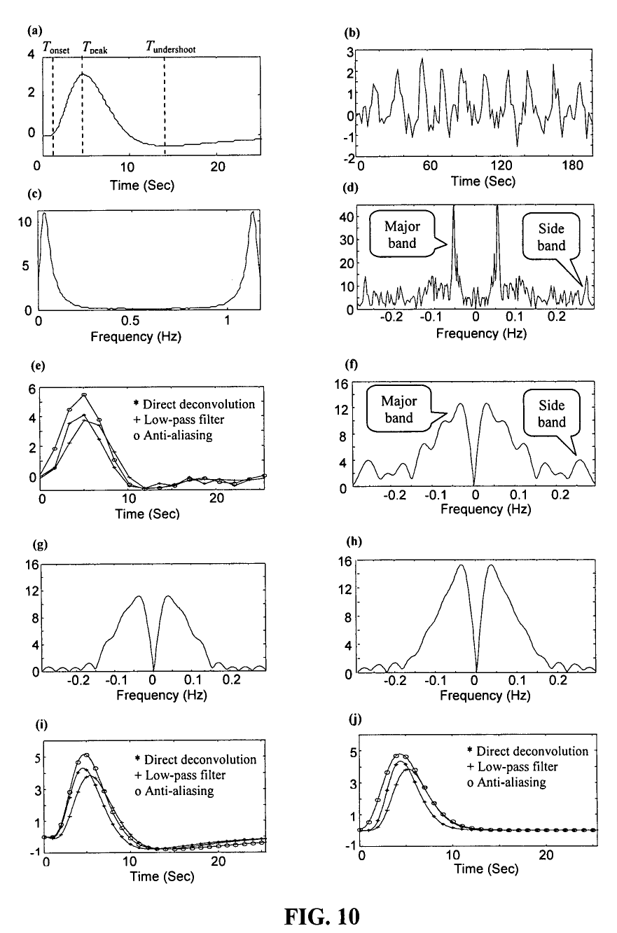

FIGS. 10A-10J show simulation results, where FIG. 10A shows a simulated HRF using a Gamma model; FIG. 10B shows a simulated BOLD signal; FIG. 10C shows the Fourier transform of the simulated HRF; FIG. 10D shows the Fourier transform of the simulated BOLD signal; FIG. 10E shows an HRF resolved from a single simulation; FIG. 10F shows the Fourier transform of the resolved HRF using direct deconvolution; FIG. 10G shows the Fourier transform of the resolved HRF using direct deconvolution with temporal smoothing; FIG. 10H shows the Fourier transform of the resolved HRF using an anti-aliasing embodiment in accordance with the subject method; FIG. 10I shows a nonlinear fitting using a Gamma model; and FIG. 10J shows a nonlinear fitting using a Gaussian model.

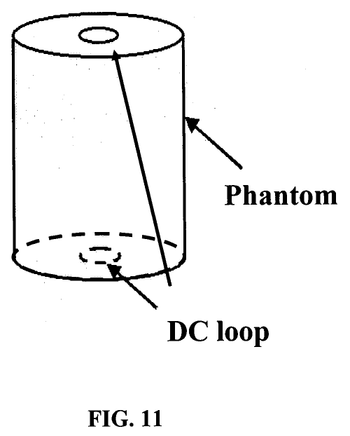

FIG. 11 shows an embodiment of a phantom having a cylindrical shape and having a loop attached to each end of the phantom such that the axis of the cylinder intersects the centers of the two loops.

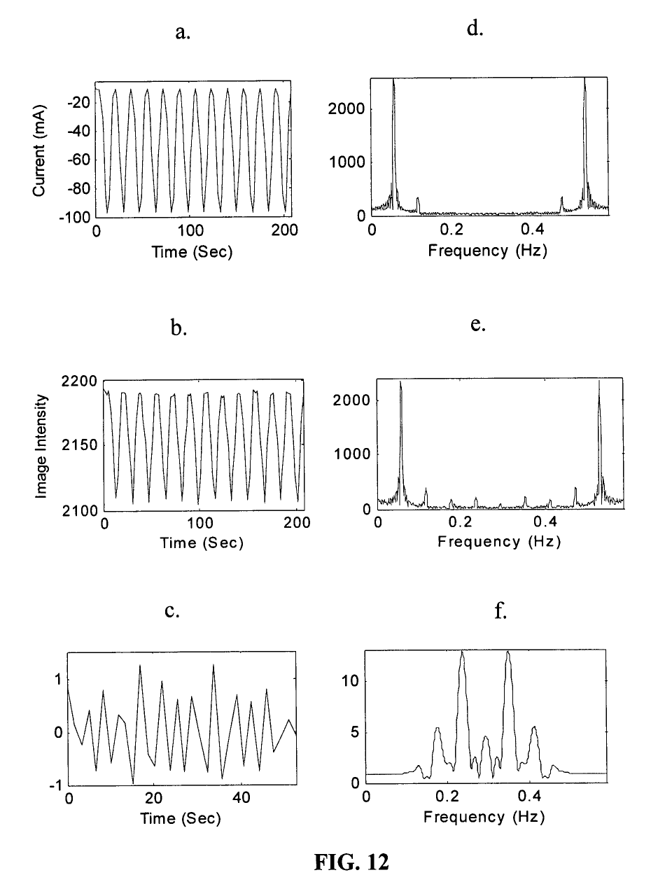

FIG. 12 shows data and results of an embodiment of the subject invention for anti-aliasing deconvolution and a determination of an aliasing transfer function for human subject #1. FIG. 12A shows time course of current signal input to BOLD simulator with aliasing removed; FIG. 12B shows time course of imaging intensity in the calibration scans; FIG. 12C shows aliasing transfer function in time domain; and FIGS. 12D-12F show Fourier transform of FIGS. 12A-12C.

### DETAILED DESCRIPTION

Embodiments of the invention relate to a method and apparatus for determined a hemodynamic response function for event-related functional magnetic resonance imaging.

An embodiment of the invention pertains to a method of determining a hemodynamic response function for event-related functional magnetic resonance imaging. The method can include locating a sample material in a region of interest in a static magnetic field B₀, such as in a MR scanner, and locating at least one coil such that wherein the at least one coil is associated with a corresponding at least one magnetic field that alters the magnetic field parallel to the static magnetic field B₀ in the region of interest. The at least one coil is then driven with a corresponding at least one time dependent current, stimₚₕₐₙₜₒₘ, where stimₚₕₐₙₜₒₘ has at least two different values. An MRI signal is then acquired by exciting the sample material with an excitation RF magnetic field that has a component perpendicular to the static magnetic field B₀, such that the magnetization of the sample material rotates and detecting a first MRI signal for the sample material, sₚₕₐₙₜₒₘ, in the region of interest. In an embodiment, the sample material is the subject. The subject is then located in the region of interest in the static magnetic field B₀. The subject is stimulated during exciting the subject with excitation RF magnetic field with a stimulation signal, stimₑᵥₑₙₜ, that is a series of substantially identical stimuli. The current, stimₚₕₐₙₜₒₘ, follows the same time course as stimₑᵥₑₙₜ. Another MRI signal is acquired by exciting the subject with excitation RF magnetic field that has a component perpendicular to the static magnetic field B₀, such that the magnetization of the subject rotates and detecting a second MRI signal for the subject, s_{BOLD}, in the region of interest. T_{system} is then determined from the relationship sₚₕₐₙₜₒₘ=stimₚₕₐₙₜₒₘ\textcircled{×}T_{system} and a hemodynamic response function, HRF, is determined from the relationship s_{BOLD}=(stimₑᵥₑₙₜ\textcircled{×}T_{system})\textcircled{×}HRF. Determining T_{system} from the relationship sₚₕₐₙₜₒₘ=stimₚₕₐₙₜₒₘ\textcircled{×}T_{system} and determining the hemodynamic response function, HRF, from the relationship s_{BOLD}=(stimₑᵥₑₙₜ\textcircled{×}T_{system})\textcircled{×}HRF can be accomplished via deconvolution. In an embodiment, stimₚₕₐₙₜₒₘ has a model HRF shape. In a further specific embodiment, stimₚₕₐₙₜₒₘ can be determined from the MRI signals. The model HRF shape can also be created from a prediction of the shape. An embodiment can use the MR scanner's gradient coils for the at least one coil.

Embodiments of the subject invention can involve a method of suppressing noise in hemodynamic deconvolution for event-related functional MR imaging (ER-fMRI). A typical ER-fMRI experiment measures the Blood Oxygenation Level Dependent (BOLD) response to a series of sparse, short-duration stimuli. Based on the deconvolution of a hemodynamic response function (HRF) from the BOLD signal and event stimulus function, the neuronal activation can be localized to specific brain regions and tracked on the order of a second. ER-fMRI can be used to study the temporal dynamics of neuronal network. However, in certain situations, aliasing noise can be generated in hemodynamic deconvolution due to the low sampling rate limited by the imaging speed. This aliasing noise can reduce the accuracy of temporal characterization of the HRF. In an embodiment, by incorporating the use of a phantom having one or more coil loops positioned perpendicular to the magnetic field Bₒ, such that DC current inputted into one of the loops will produce field distortion to Bₒ, an ER-fMRI experiment can be calibrated and the temporal measurement of HRF can be improved with the removal of aliasing noise. In an embodiment, a phantom incorporating one or more coil loops in accordance with FIG. 11 can be used to calibrate an ER-fMRI experiment, so as to improve the temporal measurement of HRF with the removal of aliasing noise.

FIG. 1 shows an embodiment of an instrumentation set up for ER-fMRI calibration using a phantom apparatus incorporating two coil loops and a spherical imaging phantom. Alternative embodiments can use sample materials with different shapes. In an embodiment, the gradient coils of the MR scanner can be used to produce fields in the B₀ direction. In further embodiments, the subject to be imaged can be used as a sample material for the initial scan.

Referring to FIG. 1, a Maxwell coil pair is placed symmetrically on the two opposite sides of a spherical imaging phantom. The coils and the phantom are both put in an RF coil, such as a birdcage coil, and aligned along the major magnetic field direction inside the MR magnet. Embodiments of the invention can use other RF coil arrays. When a current is applied in the Maxwell coil pair, a z-axis gradient inside the phantom will be produced to diphase the nuclear spins for generating a change in imaging intensity. In an embodiment, the strength of this gradient can be controlled using a computer and a control box. The imaging contrast induced by the current in the Maxwell coils can be used to simulate the BOLD contrast in fMRI experiments. In an embodiment, the maximum current in the coils is calibrated to yield an imaging contrast of about 5% at the center of the phantom relative to the basal level of imaging intensity when no current is applied in the coils. This is comparable to the maximum BOLD contrast reported in most fMRI experiments at 3 T. In an embodiment, there are 31 current levels available, which offer the capability of simulating a wide variety of BOLD signal shapes.

The plot in FIG. 2 gives a linearity test on an embodiment of a BOLD simulator in accordance with the subject invention and shows how imaging intensity in the center region of the phantom varies with the current level in the coils. In an embodiment, the phantom can be located in the center of RF coil during each imaging scan. The calibration scans can be performed before and/or after the human, or other subject, scans using the same EPI protocol and RF coil in human scans. In the calibration scans, a sequence of current levels can be input to the BOLD simulator at the same rate as that of the imaging acquisition. In an embodiment, this sequence can be generated in the following ways: 1) a BOLD-like signal is first produced by repeating a waveform of BOLD-like shape on the same time course as the event stimulus function for human subjects; 2) a sequence of numbers is then generated by sampling the BOLD-like signal at the same rate as that in the designed experiments; and 3) these numbers are finally converted to a sequence of current levels based on the linearity test result in FIG. 2 and used as the input to the BOLD simulator.

The denoising method can involve the use of a linear deconvolution model. In fMRI scans,

s_{BOLD}=stimₑᵥₑₙₜ\textcircled{×}HRF\textcircled{×}T_{system}=(stimₑᵥₑₙₜ\textcircled{×}T_{system})\textcircled{×}HRF (1)

where s_{BOLD} is the BOLD signal of a single voxel in human brain, HRF is the Hemodynamic Response Function, stimₑᵥₑₙₜ is the event stimulus function, and T_{system}, termed as aliasing transfer function in the following text, represents the influence of the imaging system on hemodynamic deconvolution. This aliasing transfer function can be determined from the calibration data using a phantom, or subject to be imaged, based on the linear transform model below:

sₚₕₐₙₜₒₘ=stimₚₕₐₙₜₒₘ\textcircled{×}T_{system} (2)

where sₚₕₐₙₜₒₘ is the time course of phantom imaging intensity and stimₚₕₐₙₜₒₘ is the sequence of current levels input to the BOLD simulator with the aliasing effects removed. In an embodiment, this sequence can be obtained by 1) sampling the generated BOLD-like signal at a rate two times of that in imaging; 2) applying a low-pass filter; 3) downsampling the sequence by a factor of two; and 4) converting to the current levels. A least-square deconvolution method can be used to resolve the aliasing transfer function from the measured time course of phantom signal and this sequence of current levels. With the aliasing transfer function, an anti-aliasing method can be utilized for HRF deconvolution based on the model in Equation (1). In an embodiment, the event stimulus function is first convolved with the aliasing transfer function, and the HRF can then be resolved from the BOLD signal and the convolution result.

An embodiment of such an anti-aliasing deconvolution is shown in FIGS. 12 and 7. FIG. 12a is the current profile input to the BOLD simulator with the aliasing effects removed. The negative sign of the current values arises from the fact that the increase of current will reduce the image intensity. FIG. 12b shows the time course of imaging intensity in the calibration scans of BOLD simulator. FIG. 12c gives the resolved aliasing transfer function. FIG. 12d-f are the Fourier Transform of the data in FIG. 12a-c. It can be seen from FIG. 12e that aliasing happens between 0.2 and 0.4 Hz, but not from FIG. 12d. The Fourier Transform of the aliasing transfer function in FIG. 12f shows that the magnitudes are large in high frequencies, which implies the aliasing mostly affect the relatively higher frequency components in BOLD signal.

FIG. 7a shows a single-voxel BOLD signal acquired from a human subject. FIG. 7b shows the stimulus function that induced this BOLD response. FIG. 7c shows the HRF resolved using a direct deconvolution method. It can be seen that the result gives the basic features of a HRF, but contains substantial noise. FIG. 7d shows the convolution of the stimulus function in FIG. 7b and the aliasing transfer function in FIG. 14c. If there were correlation between the aliasing transfer function and the noise shown in FIG. 7c, one would expect that the HRF deconvolution should be improved with the removal of the noise using this anti-aliasing method. If there were no correlation, the use of such an aliasing transfer function would generate either a HRF totally different from that in FIG. 7c or a large residue error in the algorithm. From the result shown in FIG. 7e, it can be seen that a reasonable HRF is resolved and the noise shown in FIG. 7c is reduced, if not totally removed. The ratio of sum of square error between this two deconvolution is 1.1, which implies that the aliasing transfer function does not introduce any significant uncorrelated noise and this method is very efficient in denoising for HRF deconvolution.

The subject invention pertains to the incorporation of one or more coils for producing dc magnetic fields parallel to the static magnetic field Bₒ. Such dc field coils can be used in conjunction with one or more RF coils or can be used without the RF coil(s). Preferably the dc field coil is associated with a dc field parallel to Bₒ in the region of interest. Most preferably, the dc field coil is a planar coil having a normal parallel to Bₒ. The dc field coil(s) then change the net static field experienced by the sample material located in the region of interest. Although it is described as changing the static field, it is understood that the dc current driving the dc coil can be made to vary such that the static field in the direction of Bₒ is in fact a dynamic field. In fact, the dc coil(s) can be driven with dynamically varying currents that include a model of one or more physiological functions, or other conditions to be modeled, such as patient's breathing. Again, the strength and direction of the magnetic fields associated with the dc coil(s) varies with position so that the dc coil(s) changes the field in the direction of the static field Bₒ as a function of position.

For example, if the static field coil produces a static field component in the direction parallel to Bₒ of Bₛ, then the net static field in the direction parallel to Bₒ is Bₒ+Bₛ, if B_{S} is in the same direction as Bₒ, and Bₒ-Bₛ, if B_{S} is in the opposite direction of Bo. This change in the static field can alter the T2* relaxation of by the sample material and, therefore, the resulting magnetic field B_{M} of the rotating magnetization M, where T2* characterizes the decay of the transverse component of the magnetization after excitation. A variety of sample materials can be utilized in accordance with the subject invention. The sample material can be chosen to have magnetic properties of another object or material such as a human brain, a human heart, and/or a human bone. In a specific embodiment, a gel, such as an agarose gel, can be used. The gel can incorporate substances which alter its properties. An example includes an agarose gel with copper sulfate. In this example, the concentrations of agarose and copper sulfate can be adjusted to match a particular material such as human brain tissue. In a specific embodiment, the sample material can be selected so as to have a T1 value, T2 value, and/or T1/T2 ratio in a certain range. Preferably, the sample material has a high proton density and a T2 relaxation that is long enough to provide sufficient stimulated fMRI data, where the T2 relaxation time characterizes the decay of the transverse component of the magnetization caused by spin-spin interaction after excitation. A gel having water can be used and can have additives to adjust the T2 and T1 of the gel in order to have the desired properties. In an agarose gel, copper sulfate can be used to adjust the T1. Human brain grey matter has a T1 and T2 of approximately 1000 ms and 90 ms, respectively. The sample material can also be water doped with different ions, oils, and/or organic material such as polyvinyl alcohol so as to have T1 and/or T2 comparable to a desired tissue to model, such as a human brain.

The fMRI time series signal (hemodynamic response) at time t due to neuronal activity x(t) (sensory or cognitive stimulation) is denoted by y(t), the coupling between the neuronal activity and hemodynamic response is given by

y(t)=γx(t)·h(t)+n(t) (7)

where ‘·’ denotes convolution, γ denotes the gain of the fMRI imaging process and n(t) represents the noise. The h(t) is the hemodynamic modulating function or hemodynamic “impulse response” of the BOLD signal. The h(t) has been modeled as different functions, such as a Poisson function, a Gamma function, and a Gaussian function. Therefore, these, and/or other, functions can be employed in the BOLD signal modeling for the phantom. The mathematical forms of these functions can be represented as follows:
a. Poisson function

$$h ( t ) = \frac{{λ}^{t} {e}^{- λ}}{t !} \qquad ( 8 )$$

where λ represents the lag and dispersion.
b. Gaussian function

$$h ( t ) = \frac{1}{\sqrt{2 {πσ}^{2}}} exp ( - \frac{{( t - μ )}^{2}}{{σ}^{2}} ) \qquad ( 9 )$$

where μ and σ are the mean and standard deviation (or lag and dispersion) of the function.
c. Gamma function

$$h ( t ) = \frac{{( t / τ )}^{n - 1} {e}^{- t / τ}}{τ ( n - 1 ) !} \qquad ( 10 )$$

where t is time, τ is a time constant, and n is a phase delay. A pure delay between stimulus onset and the beginning of the fMRI response is also allowed. The pure delay here accounts for any systematic asynchrony between stimulus onset and data acquisition and for any real delay between stimulus onset and hemodynamic response.

In a specific embodiment, a coil loop can be positioned perpendicular to the magnetic field B₀, such that DC current inputted into the loop will produce field distortion to B₀, where the distortion decays with distance from the loop. In other embodiments, one or more coils associated with a magnetic field having a component parallel with static field Bₒ can be utilized. The coil(s) can be positioned above, below, and/or within the sample material. The incorporation of coil(s) providing fields in the Bₒ direction can be in conjunction with the use of RF coils as described associated with magnetic fields perpendicular to Bₒ, or can be used without such RF coils. The field distortion can affect the image in two ways. The field distortion can cause a global shift of the image due to B₀ offset. The field distortion can also cause signal loss due to the gradient of field distortion in B₀ direction, which can be used for simulating BOLD effect. The global shift can be compensated for both active and basal states in BOLD imaging by, for example, using a phantom of cylinder shape with a loop attached to each end, where the axis of the cylinder intersects with the centers of the two loops. FIG. 11 shows an embodiment of the subject phantom having a cylindrical shape and having a loop attached to each end of the phantom such that the axis of the cylinder intersects the centers of the two loops. In a specific embodiment, a plurality of DC coil loops can be positioned and provided with current to produce a substantially constant B-field over the region of interest.

Referring to FIG. 11, a DC current can be input to one or both of the end loops to induce local field distortion, which simulates susceptibility effect as BOLD. Diodes can be used to control on and off states. In an embodiment, the ‘on’ state can be such that both loops have currents and the ‘off’ state can be such that only one loop has current, but of 2 times magnitude. The field of the ‘off’ state in this embodiment is more inhomogeneous in z direction than the field of the ‘on’ state, but the offsets of the z component of the magnetic field are almost the same. The embodiment shown in FIG. 11 can be operated in a mode that is T2-dependent. In theory, the coupling between the end loops and receiver coils is small and, therefore, it should not introduce much noise, which is verified by experiments.

Referring to the embodiment shown in FIG. 11, two solenoids (10-turn loops, 2 cm in diameter) were put on the ends of a phantom in accordance with the subject invention, with the axes of the end loops along B₀ direction. For the ‘on’ state, 300 mA current was driven to both solenoids, and 60 images were acquired. For the ‘off’ state, 600 mA current was driven to only one solenoid, and 60 images were acquired. Other embodiments utilizing one or more coils as shown in FIG. 11 can drive the coils with a time varying current, in order to model time-varying activities. In a specific embodiment, the coil(s) can be driven by a time-varying current that models breathing.

In an embodiment, two small loops can be placed inside an embodiment of the subject phantom. Each of the two loops can be associated with magnetic fields having a component parallel to the static field B₀ in the region of interest. In an embodiment, the two loops are planar, parallel, perpendicular to the magnetic field B₀ (in z direction), and associated with magnetic fields parallel with the B₀ field. DC current can be input to the loops to induce a local field distortion in the Bₒ field. The local field distortion can be used to simulate a susceptibility effect. Susceptibility effects can be used for imaging purposes, such as used in BOLD. An embodiment can make the field of one state more inhomogeneous in the z direction than another state and, therefore, the dephasing effect is different, resulting in a signal difference for the two states. The two loops can be driven with co-rotating currents, which can be referred as a Helmholtz pair, and/or the two loops can be driven with counter-rotating currents, which can be referred as a Maxwell pair.

In an embodiment, with the two loops driven as a Maxwell pair the current flows gives rise to little, or no, distortion of the magnetic field at the midway point between the two loops and a fairly uniform field gradient in the z direction parallel to the Bₒ field in a wide range around the midpoint. The uniform gradient of the distortion in the Bₒ field can facilitate multi-slice MRI acquisition and make the signal change insensitive to slice position.

An embodiment of a phantom can incorporate a sample material having a cylindrical shape. The susceptibility effect due to the sample material in a phantom is added to the B₀ distortion induced by the DC currents in the, for example, two loops, and gives rise to a complex pattern of signal change depending on the current direction and slice position. In an embodiment, the shape of the sample material can be controlled to control B₀ distortion within the phantom. In an embodiment, the shape of the sample material is selected to achieve a constant B₀ distortion across the ROI. In a specific embodiment, a spherical shaped sample material can be incorporated with the phantom. In theory, the spherical shaped sample material induces a constant B₀ distortion within the phantom, such that there is no additional field gradient from the sample material in the ROI.

In an embodiment, a coaxial cable can be used to connect the two loops so that the DC paths produce negligible magnetic field due to the currents canceling each other. RF chokes can be incorporated with the loop pair to minimize induced RF current. For this two loop coil structure driven as a Maxwell coil the image intensity is related to the echo time (TE), similar to BOLD imaging. In an embodiment, there is no need to decouple the loops during transmit. The field change for this design is global such that the effect can be sensitive to slice thickness and can be sensitive to slice position.

### EXAMPLE 1

In a specific embodiment, a BOLD simulator, SMARTPHANTOM™ (Invivo Diagnostic Imaging, Gainesville, Fla. 32608), was constructed and a calibration protocol for ER-fMRI experiments was developed. In phantom scans, the aliasing due to the undersampling in the imaging system can be calibrated. In human scans, a correlation between the aliasing and the noise in the HRF deconvolution for ER-fMRI data was observed. Based on the phantom calibration, an anti-aliasing embodiment in accordance with the subject method can be used to suppress the noise and improve HRF estimation at a low sampling rate. Simulations have been performed to quantitatively evaluate the anti-aliasing method in terms of the accuracy in the temporal characterization of HRF.

In an embodiment, an aliasing transfer function can be introduced in an HRF deconvolution model to correct the influence of the imaging system on the ER-fMRI data analysis when undersampling is used. This model can be written as

s(t)=f(t)\textcircled{×}h(t)\textcircled{×}T(t)+n(t)=(f(t)\textcircled{×}T(t))\textcircled{×}h(t)+n(t), (2)

where T(t) is the aliasing transfer function, and the other terms are the same as in Equation 1. It should be noted that this is still a LTI system, but the nonlinear and time-variant effects due to the imaging system can be represented by T(t), which can be calibrated using, for example, a BOLD simulator.

Imaging experiments were performed using a standard quadrature volume head coil on a SIEMENS Allegra 3T system. A T*₂-weighted single-shot gradient-echo EPI protocol was used to acquire the time course data in both human and phantom scans.

FIG. 1 shows a phantom calibration setup using a specific BOLD simulator, SMARTPHANTOM™. A Maxwell coil pair was placed symmetrically on the two opposite sides of a spherical imaging phantom (NiCl₂*H₂O in solution of H₂O, General Electronics, Milwaukee, Wis. 53201). The coils and the phantom were inserted into the RF coil and aligned along the major magnetic field direction inside the magnet. When a current is applied to the Maxwell coil pair, a z-axis gradient inside the phantom is produced. This gradient dephases the nuclear spins and generates a change in the imaging intensity. The strength of this gradient can be controlled by changing the current level in the Maxwell coils. The imaging contrast induced by the gradient can be used to simulate the BOLD contrast in a fMRI experiment. The maximum current in the coils was set to be 116 mA such that the highest imaging contrast generated at the center of the phantom was about 5% of the base line image intensity when no current was applied in the coils. This is comparable to the maximum BOLD contrast reported in most fMRI experiments at 3 T. There were a total of 31 discrete current levels in the coils. This discretization can introduce some simulation noise, the amplitude level of which is about ±0.08% of the base line image intensity. In fMRI experiments, the reported average signal to noise ratio is about 50 and contrast to noise ratio is about 2 (Wu et al., 2005). These numbers correspond to a noise amplitude level of more than ±14% of the base line image intensity, which is much larger than the simulation noise level. Therefore, this BOLD simulator is sufficiently accurate in the BOLD contrast simulation.

A linearity test was performed using the setup in FIG. 1. The current in the BOLD simulator was gradually increased from 0 to 116 mA each time by one current level. At each current level, the phantom was scanned and the mean image intensity at the center of the phantom (5 by 5 pixels) was measured. The image intensity contrast generated at a current level was defined as the difference between the image intensity at the particular current level and the base line image intensity. This test was repeated 50 times (about half an hour in total). The plot of the image intensity contrast against the current level is presented in FIG. 2 and it can be seen that the relationship is linear. This relationship makes it possible to simulate a BOLD signal by controlling the current during imaging. During the imaging scan, the phantom can be positioned at the center of the RF coil for optimum performance.

Slow ER-fMRI experiments on human subjects were performed. Eight healthy adults (subject #1-8) were asked to read non-words loudly and scanned. During each 3.5 min run, a series of 123 whole-brain EPI images were acquired with FOV=240 mm, matrix 64×64, FA=70°, TE=25 ms, and TR=1700 ms. Each whole-brain image had 32 saggital slices with a slice thickness of 5 mm and a slice gap of 5 mm. There were 5 runs with 10 non-words per run, counterbalanced across the runs for phonotactic probability. Every non-word was shown on a screen for 5.1 s and the screen was kept dark between every two consecutive non-words. The interstimulus intervals had a mean of 21 s and were jittered within a 3.4 s range to remove the influence of the HRF overlaps on HRF deconvolution.

These ER-fMRI experiments allow the simultaneous acquisition of BOLD response in vision and motor cortexes, which are the most frequently studied functional regions in fMRI. The deconvolution from these BOLD responses can give typical HRFs with clear peak and post-stimulus features. Values typically used in fMRI at 3 T, were used to set up the data acquisition time and the sampling rate. The use of these parameters can cause the undersampling in ER-fMRI data acquisition. In an embodiment, the undersampling can be reduced, or eliminated, using the HRF deconvolution model described in Eq. (2).

In an embodiment, two calibration scans are performed using the BOLD simulator, one before and one after every human subject scan in ER-fMRI data acquisition. In a specific embodiment, during each 3.5 min scan, a series of 123 EPI images can be acquired with the same parameters as those in the human scan. Each EPI volume image had 25 axial slices with a slice thickness of 5 mm and slice gap of 5 mm. The current in the BOLD simulator was changed before every image acquisition and held constant during the acquisition. Corresponding to the 123 volume images, there were 123 current values, which were determined in the following way: first, a BOLD-like signal was “artificially” produced by repeating a waveform of HRF-shape on the same time course as the event stimuli for human subjects (the appearance of non-words); second, 123 data points were acquired by sampling the BOLD-like signal at the same rate as was done in the ER-fMRI experiments; finally, the sampled data were scaled and converted to the 123 current values based on the linearity test plot in FIG. 2. In the generation of the BOLD-like signal, it is preferred to use a HRF-shape waveform as close to the real one as possible because this may affect the calibration of the aliasing transfer function.

In a specific embodiment, the HRF-shape waveform can be extracted from the real data collected in the ER-fMRI experiments. In an embodiment, a number of activated voxels can be selected. In a specific embodiment, 50 activated voxels with strong BOLD signals were selected randomly from the vision and motor cortexes of eight human subjects. A low pass filter with a cut-off frequency of 0.2 Hz was applied to these BOLD signals to remove the high frequency noise. A direct deconvolution method was used to resolve the HRF for each BOLD signal. An optimum HRF waveform was obtained by averaging the 50 resolved HRFs. The nonlinear fitting method was used to fit this waveform using the sum of two gamma functions (Friston et al., 1998). FIG. 3 shows the data points for the model fitting and the result of the fit. In an embodiment, this fitted function can be used as a standard HRF, which was described by the following equation

HRF(t)=0.17(t)⁵e⁻¹−1.3(t/5.0)²e^{-t/5.0}. (3)

### A 21 s HRF waveform with a sampling interval of 1700 ms was calculated from this equation and used as the HRF-shape waveform to generate the BOLD-like signal.

The phantom calibration experiment can be described by the following mathematical model

s(t)=I(t)\textcircled{×}T(t)+n(t), (4)

where T(t) is the aliasing transfer function for calibration, n(t) is the noise, and I(t) is the current input to the BOLD simulator, which is a scaled version of the generated BOLD-like signal. The output, s(t) is the measured BOLD-like signal, the discrete version of which were acquired by averaging the two measured time courses of the mean image intensity at the center of the phantom (5 by 5 pixels) in the two calibration scans.

It should be noted that the measured BOLD-like signal was affected by the imaging system while the current input signal was not. This provides the possibility to calibrate the influence of the imaging system on the ER-fMRI data acquisition. However, this calibration relies on the processing of the discrete signals. Apparently, a directly sampled version of I(t) would introduce the same aliasing effects as what was introduced by the imaging system in the discrete version of s(t). Hence, to calibrate the aliasing effects in undersampling, a discrete version of I(t) without aliasing was needed. A mathematical method was used to calculate this discrete version of I(t), based on the fact that the continuous version of the current signal was known. The continuous current signal was first sampled with a small sampling interval of 425 ms, which was one fourth of that required in the experiment. The sampled data were then filtered using a digital low-pass filter (designed in MATLAB®) with a cut-off frequency equal to the half of the required sampling frequency. Finally, the data were down-sampled by a factor of 4 and used as the discrete version of the current input signal I(t). Because the low-pass filter removed the high frequency components that could alias back into the low frequencies, there was no aliasing in the discrete version of I(t). With the discrete versions of I(t) and s(t), the deconvolution based on Eq. (4) was implemented to determine the discrete version of the aliasing transfer function T(t).

ER-fMRI data were processed using AFNI (The Medical College of Wisconsin and the National Institutes of Health) and MATLAB®. Registration and motion correction were implemented by minimizing the intensity difference from the first image. A HRF of 27 s long was resolved from the BOLD signal of every voxel in eight human subjects. Two deconvolution methods were used. The first method was the direct deconvolution based on Eq. (1). The event stimulus function in this method was a series of binary number, in which one represented the onset of the event and zero represented the rest state. A linear regression model was used to fit HRF and a 2^{nd} order polynomial model was used to fit the base line. Two model fittings were implemented in the data; one uses both HRF and base-line models, and the other one uses only the base-line model. F-statistics were calculated for each voxel based on the ratio between the two residual sum of squares in the model fittings. A correlation factor was given to indicate how well the data is correlated to the event stimulus function. The second method was an anti-aliasing method based on the system model in Eq. (2). F-statistics were also calculated. However, the input signal in the deconvolution was the convolution of the event stimulus function and the aliasing transfer function resolved from the phantom calibration data collected before and after each human scan. The comparison of the two HRF deconvolution methods was made to determine whether noise is introduced in the HRF deconvolution based on the model in Eq. (1) and whether the noise is suppressed if the model in Eq. (2) is used.

To quantitatively evaluate the anti-aliasing method in comparison to other HRF deconvolution methods, simulations were performed using the model in FIG. 4. In the simulation, only the aliasing effects correlated to the data truncation and the undersampling were considered and the imaging system was approximated by an all pass LTI system. The same event stimulus function as the one used in ER-fMRI experiments was used as the stimuli. The HRF was calculated from Eq. (3). Random noise of Gaussian distribution was generated in MATLAB®. To simulate the aliasing effects, the data was first generated with the sampling interval of 425 ms and the BOLD signal was down-sampled by a factor of 4 at the final stage. The SNR of the simulated BOLD signal was measured and compared with a real BOLD signal collected from subject #1. The power of the random noise was adjusted until the two SNRs were comparable. In this simulation, three deconvolution methods were investigated; the direct deconvolution method based on Eq. (1), the direct deconvolution method with the temporal smoothing of BOLD signals, and an embodiment of the subject anti-aliasing method. In the second method, a low-pass filter was applied to the BOLD signal to suppress the aliasing effects before the deconvolution. The comparison between this method and the anti-aliasing method was made to determine whether the latter was more efficient than a simple temporal smoothing method in terms of the temporal characterization of HRF.

The simulation described above was performed for 10,000 times with different random noise generation and the same SNR. The HRF deconvolution was applied to each simulated data respectively using three different methods. A nonlinear fitting was used to fit the resolved HRFs based on a Gamma fitting model

γ(t)=K₁(t/τ₁)⁶e^{-t/τ}^{₁}+K₂(t/τ₂)²e^{-t/τ}^{₂}, (5)

and a Gaussian fitting model

$$G ( t ) = K exp [ - 0.5 * {( \frac{t - μ}{σ} )}^{2} ] . \qquad ( 6 )$$

Apparently, the Gamma model is a good fit for the simulated HRF described by Eq. (3) and the Gaussian fitting model does not result in a good fit. The use of two different models simulates two situations in HRF fitting; 1) when the fitting model matches the real HRF, and 2) when the data fit is inadequate. In the real HRF timing analysis, either situation may occur, since the real HRF may be unknown; and then different nonlinear fitting models can be selected. This comparison gives a quantitative analysis on how different HRF deconvolution method can affect the HRF timing analysis in different situations. With the nonlinear fitting results from all the simulations, three temporal parameters, time-to-onset, time-to-peak, and time-to-undershoot, were statistically measured to quantitatively evaluate three deconvolution methods in terms of the accuracy of temporal characterization of hemodynamic response in ER-fMRI.

FIG. 5 gives a real BOLD signal acquired from subject #1 in a 3.5 min ER-fMRI run with a sampling interval of 1700 ms. FIG. 5(a) is a BOLD signal acquired from subject #1. FIG. 5(b) is its Fourier transform. FIG. 5(c) gives the HRF resolved from this signal using the direct deconvolution method. FIG. 5(d) is the Fourier transform of the HRF. From FIGS. 5(b) and 5(d), it can be seen that the frequency spectra of the BOLD signal and the HRF have similar features. There are a strong major band below 0.1 Hz and some small side bands above 0.1 Hz. These side-band components, especially those strong ones above 0.2 Hz, are not the features of a BOLD signal or a HRF. In the following data analysis, it will be demonstrated that these features are correlated to truncation and undersampling of the data.

FIG. 6 shows how an aliasing transfer function is resolved from the phantom calibration data collected before and after the scan on the human subject #1. FIG. 6 (a)-(c) are the time domain representation and (d)-(f) are the Fourier transforms with the zero-frequency components removed. FIG. 6(a) is the discrete version of the current input to the BOLD simulator without aliasing effects. The negative sign of the current values arises from the fact that the increase of current will reduce the image intensity. FIG. 6(b) shows the average time-course of the imaging intensity in the two calibration scans on the BOLD simulator. The aliased side-band signals can be clearly seen above 0.2 Hz in FIG. 6(e), but not in FIG. 6(d). The resolved aliasing transfer function is given in FIG. 6(c) and its Fourier transform in FIG. 6(f). It was observed that the frequency spectrum of this aliasing transfer function has higher magnitudes at higher frequencies, which implies that more aliasing occurs at higher frequencies in the BOLD signal. The imaging system is not time-invariant and its performance often varies at a very slow speed. This instability may affect the determination of an aliasing transfer function. Strictly speaking, the aliasing transfer function cannot be defined as a time-invariant function. There exists an approximation in Eq. (2). This approximation reduces the complexity of the system model and makes the calibration feasible. To minimize the time variance due to system instability, it is preferred that the phantom calibrations should be made right before and after every human scan. In the following data analysis, it will be demonstrated that the aliasing related noise can be effectively suppressed under this approximation and using this calibration method.

The data acquired from eight human subjects were processed and the HRF of every single voxel was resolved using both the direct deconvolution method and the anti-aliasing method. FIG. 7 shows an example; FIG. 7(a) shows a single-voxel BOLD signal acquired from subject #1, FIG. 7(b) shows the event stimulus function that induced this BOLD response. And FIG. 7(c) shows the HRF resolved using the direct deconvolution method. It can be seen that the result gives the basic features of a HRF, but contains considerable noise. FIG. 7(d) shows the convolution of the event stimulus function in FIG. 7(b) and the aliasing transfer function in FIG. 6(c). FIG. 7(e) gives the HRF resolved using the anti-aliasing method. If there were correlation between the aliasing transfer function and the noise shown in FIG. 7(c), one would expect that the use of aliasing transfer function in the anti-aliasing method should remove that noise. This is indeed the result shown in FIG. 7(e).

More deconvolution results from different brain regions and human subjects are shown in FIG. 8. In comparison to those using the direct deconvolution, the HRFs resolved using an embodiment of the anti-aliasing method in accordance with the subject invention are qualitatively better. The noise in the hemodynamic deconvolution using the direct deconvolution method was significantly suppressed in the anti-aliasing method. A quantitative analysis of the noise suppression was also made and the results are shown in FIG. 9 and Table 1. In this analysis, the activated voxels that have a high correlation factor (>0.5) in F-statistics were selected from the vision and motor cortexes of each subject. The BOLD signals acquired from these voxels were truncated to generate six data sets of different time length from 110 s to 210 s. The HRF deconvolution was applied to each set of data respectively using the direct deconvolution and the anti-aliasing method.

Because the noise due to the data truncation and aliasing was mostly within the frequency band above 0.2 Hz, the power above 0.2 Hz in HRF was calculated and the ratio of this calculated power to the total power of HRF was used as a measure of the noise level in HRF deconvolution. This noise level was measured in every HRF and the mean value and standard deviation were calculated for every subject. FIG. 9 gives the plot of the noise level versus the data length for every subject. In the direct deconvolution method, it can be seen that the noise level decreases as the data length increases. In the anti-aliasing method, it can be seen that the noise level is always low.

The correlation coefficients between the noise level in HRFs and the data length are shown in Table 1. The values of the coefficients are high in the direct deconvolution method while low in the anti-aliasing method. This demonstrates the correlation between the noise in HRF deconvolution and the data truncation. Also, the use of the anti-aliasing method can significantly reduce this correlation, which demonstrates that the correlation between the aliasing transfer function and the noise in HRF deconvolution, and further adds support to the system model in Eq. (2). All of these results suggest that the imaging system acts as a noise source to the fMRI experiments and the generation of this noise is related to the data truncation and limitation in sampling frequency. It should also be noted that the results in FIG. 9 suggest that this noise can be low if the data acquisition time is sufficiently long. However, a long experiment can also reduce the data reliability as the discomfort of the human subjects increases with the time in the magnet.

Simulation results are given in FIG. 10. FIG. 10(a) shows the simulated HRF using the model in Eq. (3) with the sampling interval of 425 ms. A simulated BOLD signal of 3.5 min long with a sampling interval of 1700 ms is given in FIG. 10(b). The frequency spectra of the simulated HRF and the BOLD signal are shown in FIG. 10(c) and FIG. 10(d). It can be seen that the real HRF is within a low and narrow frequency band, but the BOLD signal that is acquired by down-sampling and data truncation has side bands above 0.2 Hz due to aliasing. This simulated result agrees well with the experimental data shown in FIG. 5(b). Three HRFs were resolved from the simulated BOLD signal in FIG. 10(b) respectively, using three different methods; direct deconvolution, direct deconvolution with temporal smoothing, and the anti-aliasing method with an aliasing transfer function given in FIG. 6(c). FIG. 10(e) shows the resolved HRFs.

It can be seen that the direct deconvolution introduces considerable noise. This noise can be removed by temporal smoothing in the second method or by anti-aliasing method. FIGS. 10(f), 10(g) and 10(h) show the frequency spectra of the resolved HRFs in FIG. 10(e) respectively. The presence of strong side-band signals above 0.2 Hz can be seen in the HRF, resolved using the direct deconvolution. This result agrees well with the experimental data in FIG. 5(d). The removal of the side-band signals in FIGS. 10(g) and 10(h) further demonstrates the correlation between the noise in the direct deconvolution and the high-frequency side bands. In FIG. 10(e), some amplitude difference can be seen in HRFs resolved using the three different methods. This is related to whether and how a convolution is implemented before the deconvolution step in the above methods. In the anti-aliasing method, the event stimulus function is first convolved with an aliasing transfer function, which reduces the power of the input in the deconvolution and increases the gain of the resolved HRF. In the direct deconvolution with the temporal smoothing, the BOLD signal is first passed through a low-pass filter, which reduces the output power in the deconvolution and the gain of the resolved HRF as well. It should be noted that the aliasing transfer function offers a means to calibrate the gain of the imaging system, which can help to compare the fMRI experiments performed on different MRI scanners or at different times.

FIG. 10(i) gives the averaged nonlinear fitting results from 10,000 simulations using Gamma model in Eq. (5) and FIG. 10(j) gives those using Gaussian model in Eq. (6). Table 2 gives the statistical measurements of three temporal parameters on all the simulations. The direct fitting results given in the table are obtained from the measurement data by directly fitting the HRF in Eq. (3), using the models in Eqs. (5) and (6). This gives the information about how much bias is introduced by the mismatch of the fitting model. When this bias is small (Gamma Model), which means the fitting model matches the real HRF well; it can be seen that the anti-aliasing method gives the highest accuracy in the measurements of all the temporal parameters, because of the efficient suppression of the aliasing-related noise. Using the direct deconvolution method, there exists a large error in the estimation on the time-to-undershoot, which implies that the aliasing-related noise introduces substantial distortion in the undershoot region of the HRF. The temporal smoothing can reduce this distortion and can improve the accuracy of this measurement. However, the payback is that the temporal smoothing can also affect the peak and onset region of the HRF and can reduce the accuracy of the measurements of time-to-peak and time-to-onset. When the fitting model does not sufficiently match the real HRF (Gaussian model), it can be seen that all the three methods show poor performance. In this case, the mismatch dominates the error in the measurements and even the direct fitting is not capable of giving a good estimate on some temporal parameters.

The data truncation and undersampling in ER-fMRI experiments can introduce aliasing in BOLD signal acquisition. Due to aliasing, preferably, the imaging system is not neglected in the system model for hemodynamic deconvolution. An aliasing transfer function can be resolved and used to suppress the aliasing-related noise in hemodynamic deconvolution. Using simulation, it is quantitatively demonstrated that this calibration method can efficiently improve the accuracy of the temporal characterization of hemodynamic response for BOLD ER-fMRI analysis, when the nonlinear fitting model matches the real HRF.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

### REFERENCES

Boynton, G. M., Engel, S. A., Glover G. H., Heeger D. J., 1996. Linear systems analysis of functional magnetic resonance imaging in human VI. J. Neurosci. 16, 4207-4221.
Buckner, R. L., Bandettini, P. A., O'Craven, K. M., Savoy, R. L., Petersen, S. E., Raichle, M. E., and Rosen, B. R., 1996. Detection of cortical activation during averaged single trials of a cognitive task using functional magnetic resonance imaging. Proc. Natl. Acad. Sci. USA 93, 14878-14883.
Brown, G. G., Vangel, M. G., Kikinis, R., Wells, W. M., 2005. Reproducibility of functional MR imaging: Preliminary results of prospective multi-institutional study performed by biomedical informatics research network. Radiology 237, 781-789.
Buckner, R. L., 1998 Event-related fMRI and the hemodynamic response. Hum. Brain Mapping 6, 373-377.
Burock, M. A., Dale, A. M., 2000. Estimation and detection of event-related fMRI signals with temporally correlated noise: a statistically efficient and unbiased approach. Hum. Brain Mapping 11, 249-260.
Buxton, R. B., Wong, E. C., Frank, L. R., 1998. Dynamic of blood flow and oxygenation changes during brain activation: the balloon model. Magn. Reson. Med. 39, 866-864.
Buxton, R. B., Uludag, K., Dubowitz D. J., Liu, T. T., 2004. Modeling the hemodynamic response to brain activation. NeuroImage 23, S220-S223.
Dale, A. M., 1999. Optimal experimental design for event-related fMRI. Hum. Brain Mapping 8, 109-114.
Dilharreguy, B., Jones, R. A., Moonen, C. T. W., 2003. Influence of fMRI data sampling on the temporal characterization of the hemodynamic response. NeuroImage 19, 1820-1828.
Duann, J., Jung, T., Kuo, W., Yeh, T., Makeig, S., Hsieh, J., Sejnowski, T. J., 2002. Single-trial variability in event-related BOLD signals. NeuroImage 15, 823-835.
Ernst, T., Hennig, J., 1994. Observation of a fast response in functional MR. Magn. Reson. Med. 32, 146-149.
Friston, K. J., Fletcher, P., Josephs, O., Holmes, A., Rugg, M. D., Turner, R., 1998. Event-related fMRI: Characterizing differential responses. NeuroImage 7, 30-40.
Friston, K. J., Josephs, O., Rees, G., Turner, R., 1998. Non-linear event-related responses in fMRI. Magn Reson Med 39, 41-52.
Gitelman, D. R., Penny, W. D., Ashbumer, J., Friston, K. J., 2003. Modeling regional and psychophysiologic interactions in fMRI: the importance of hemodynamic deconvolution. NeuroImage 19, 200-207.
Glover, G. H., 1999. Deconvolution of impulse response in event-related BOLD fMRI. NeuroImage 9, 416-429.
Handwerker, D. A., Ollinger, J. M., D'Esposito, M., 2004. Variation of BOLD hemodynamic response across subjects and brain regions and their effects on statistical analyses. NeuroImage 21, 1639-1651.
Hu, X., Le, T. H., Ugurbil, K., 1997. Evaluation of the early reponse in fMRI in individual subjects using short stimulus duration. Magn. Reson. Med. 37, 877-884.
Kershaw J, Kashikura K, Zhang X, Abe S, Kanno I, 2001. Bayesian Technique for Investigating Linearity in Event-related BOLD fMRI. Magn Reson Med 45, 1081-1094.
Kruggel, F., von Cramon, D. Y., 1999. Temporal properties of the hemodynamic response in functional MRI. Hum. Brain Mapping 8, 259-271.
Liu, T. T., Frank, L. R., Wong, E. C., Buxton, R. B., 2001. Detection power, estimation efficiency, and predictability in event-related fMRI. NeuroImage 13, 759-773.
Liu, J. Z., Zhang, L., Brown, R. W., Yue, G. H., 2004. Reproducibility of fMRI at 1.5 T in a strictly controlled motor task. Magn. Reson. Med. 52, 751-760.
Lu, Y., Jiang, T., 2005. Single-trial variable model for event-related fMRI data analysis. IEEE Trans. Med. Imaging 24, 236-245.
Maccotta, L., Zacks, J. M., Buckner, R. L., 2001. Rapid self-paced event-related functional MRI: feasibility and implications of stimulus-versus response-locked timing. NeuroImage 14, 1105-1121.
Menon, R. S., Strupp, J. P., Anderson, P., Ugurbil, K., 1995. BOLD based functional MRI at 4 tesla includes a capillary bed contribution: echo-planar imaging correlates with previous optical imaging using intrinsic signals. Magn. Reson. Med. 33, 453-459.
Menon, R. S., Luknowsky, D. C., Gati, J. S., 1998. Mental chronometry using latency-resolved functional magnetic imaging. Proc. Natl. Sci. USA 95, 10902-10907.
Miezin, F. M., Maccortta, L., Ollinger, J. M., Peterson, S. E., Buckner R. L., 2000. Characterizing the hemodynamic response: effects of presentation rate, sampling procedure, and the possibility of ordering brain activity based on relative timing. NeuroImage 11, 735-759.
Neumann, J., Lohmann, G., Zysset, S., Cramon, D. Y., 2003. Within-subject varability of BOLD response dynamics. NeuroImage 19, 784-796.
Oppenheim, A. V., Willsky, A. S., Hamid S., Nawab S. H., 1996. Signals and Systems. Prentice Hall, New Jersey.
Richter, W., Andersen, P. M., Georgopoulos, A. P., Kim, S. G., Rosen, B. R., 1997. Sequential activity in human motor areas during a delayed cued finger movement task studied by time-resolved fMRI. NeuroReport 8, 1257-1261.
Rosen, B. R., Buckner, R. L., Dale, A. M., 1998. Event-related functional MRI: Past, present and future. Proc. Natl. Acad. Sci. USA 95, 773-780.
Tan, A. H., Godfrey, K. R., 2002. The generation of binary and near-binary pseudorandom signals: an overview. IEEE Trnas. on Instrumentation and Measurement 51, 583-588.
Van Gelderen P., Wu, C. W. H., de Zwart J. A., Cohen, L., Hallett, M., Duyn, J. H., 2005. Resolution and reproducibility of BOLD and perfusion functional MRI at 3.0 Tesla. Magn. Reson. Med. 54, 569-576.
Vazquez, A. L., Noll, D. C., 1998. Nonlinear aspects of the BOLD response in functional MRI. NeuroImage 7, 108-118.
Wu, G., Li, S., 2005. Theoretical noise model for oxygenation-sensitive magnetic resonance imaging. Magn. Reson. Med. 53, 1046-1054.
Zarahn, E., 2000. Testing for neural response during temporal components of trials with BOLD fMRI. NeuroImage 11, 655-666.
Zou, K. H., Greve, D. N., Wang M., Pieper, S. D., Warfield, S. K., White, N. S., Manandhar, S.,
Brown, G. G., Vangel, M. G., Kikinis, R., Wells, W. M., 2005. Reproducibility of functional MR imaging: Preliminary results of prospective multi-institutional study performed by biomedical informatics research network. Radiol. 237, 781-789.

1. A method of determining a hemodynamic response function for event-related functional magnetic resonance imaging, comprising:
locating a sample material in a region of interest in a static magnetic field B₀;
locating at least one coil, wherein the at least one coil is associated with a corresponding at least one magnetic field that alters the magnetic field parallel to the static magnetic field B₀ in the region of interest;
driving the at least one coil with a corresponding at least one time dependent current, stimₚₕₐₙₜₒₘ, where stimₚₕₐₙₜₒₘ has at least two different values;
exciting the sample material with an excitation RF magnetic field that has a component perpendicular to the static magnetic field B₀, such that the magnetization of the sample material rotates;
detecting a first MRI signal for the sample material, sₚₕₐₙₜₒₘ, in the region of interest;
locating a subject in the region of interest in the static magnetic field B₀;
stimulating the subject during exciting the subject with excitation RF magnetic field, wherein the stimulation signal, stimₑᵥₑₙₜ, is a series of substantially identical stimuli, wherein stimₚₕₐₙₜₒₘ follows the same time course as stimₑᵥₑₙₜ;
exciting the subject with excitation RF magnetic field that has a component perpendicular to the static magnetic field B₀, such that the magnetization of the subject rotates;
detecting a second MRI signal for the subject, s_{BOLD}, in the region of interest;
determining T_{system} from the relationship sₚₕₐₙₜₒₘ=stimₚₕₐₙₜₒₘ\textcircled{×}T_{system}; and
determining a hemodynamic response function, HRF, from the relationship s_{BOLD}=(stimₑᵥₑₙₜ\textcircled{×}T_{system})\textcircled{×}HRF.

2. The method according to claim 1, wherein determining T_{system} from the relationship sₚₕₐₙₜₒₘ=stimₚₕₐₙₜₒₘ\textcircled{×}T_{system} comprises deconvolution.

3. The method according to claim 1, wherein determining a hemodynamic response function, HRF, from the relationship s_{BOLD}=(stimₑᵥₑₙₜ\textcircled{×}T_{system})\textcircled{×}HRF comprises deconvolution.

4. The method according to claim 1, wherein stimₚₕₐₙₜₒₘ has a model HRF shape.

5. The method according to claim 1, wherein locating a sample material in a region of interest comprises locating the sample material in an MR scanner wherein the at least one coil comprises the MR scanner's gradient coils.

6. The method according to claim 1, wherein the sample material is spherical and the at least one coil comprises a first coil adjacent a first side of the sample material.

7. The method according to claim 6, wherein the at least one coil comprises a second coil adjacent a second side of the sample material, wherein the second side is opposite the first side.

8. The method according to claim 1, wherein the sample material is the subject.

9. The method according to claim 1, wherein the subject is a human being.

10. The method according to claim 4, wherein the model HRF shape is determined from the first and second MRI signals.
